(19) United States
(12) Patent Application Publication
(10) Pub. No.: US 2009/0018780 A1
(43) Pub. Date: Jan. 15, 2009

(54) Hazardous Substance Release Notification System

(76) Inventor: Yoganand John Sookhu, Englewood, NJ (US)

Correspondence Address: Ashok Tankha, 36 Greenleigh Drive, Sewell, NJ, 08080, US

(21) Appl. No.: 12/109,358

(22) Filed: Apr. 25, 2008

Related U.S. Application Data
(60) Provisional application No. 60/926,253, filed on Apr. 26, 2007.

(51) Int. Cl. G06F 19/00; G01N 33/00; G06F 15/00

(52) U.S. Cl. 702/24; 73/31.02; 702/188

(57) ABSTRACT

Disclosed herein is a method and system for obtaining information on contaminants in ambient air. Multiple detection systems sample the ambient air for the contaminants in real time. Each of the multiple detection systems analyzes contaminants for hazardous substances. The multiple detection systems transmit information on the hazardous substances to a satellite monitoring system. The satellite monitoring system receives the transmitted information. The satellite monitoring system packages the information transmitted from each of the detection systems and analyzes the packaged information. The satellite monitoring system transmits the analyzed information to a console deployed on a computing device at a command station. The console receives the transmitted information from the satellite monitoring system and communicates user interaction based on the transmitted information to the satellite monitoring system.

### CROSS REFERENCE TO RELATED APPLICATIONS

This application claims the benefit of provisional patent application number U.S. “60/926,253” titled “Hazardous Substance Release Notification System”, filed on “Apr. 26, 2007” in the United States Patent and Trademark Office.

### BACKGROUND

This invention, in general, relates to notification systems. More particularly, this invention relates to obtaining information on contaminants in ambient air.

In the period prior to the 9-11 attacks, one of the primary focuses of law enforcement agencies was to address the booming drug trafficking trade. In a post 9-11 society, preventing terrorist or other threats related to the release of hazardous substances has been given higher priority by law enforcement agencies.

In the post 9-11 society, people's complacency has been replaced with a heightened sense of awareness of the surroundings and potential enemies. After the 9-11 attacks on American soil, law enforcement agencies have focused their efforts to identify potential and future attacks. Since this was a scenario unfamiliar to the American society, the situation was addressed by adding thousands of personnel and millions of extra man hours to protect our nation. As a result, public gathering places, ports, and roadways become human bottlenecks and daily routines are severely impacted. Another negative effect of these actions is allegations of racial profiling of certain ethnic groups. The increased security also raised the consciousness of the public about government encroachment on their privacy.

Therefore, there is a need for a method and system for identification and mitigation of an accidental or intentional release of hazardous substances, for example, harmful chemicals, radioactive and biological substances, illegal drugs, etc. There is also a need for the method and system to identify and mitigate the accidental or intentional release of the hazardous substances without encroaching people's privacy.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The method and system disclosed herein addresses the above stated need for identifying and mitigating accidental or intentional release of hazardous substances, for example harmful chemicals, radioactive or biological substance, illegal drugs, etc. in real time using a hazardous substance release notification system capable of publishing findings of hazardous substances in real time via wireless technologies.

Disclosed herein is a method and system for obtaining information on contaminants in ambient air. Multiple detection systems sample the ambient air for the contaminants in real time. The detection systems analyze the contaminants for hazardous substances. The detection systems transmit information on the hazardous substances to a satellite monitoring system. The satellite monitoring system receives the transmitted information. The satellite monitoring system packages the transmitted information and analyzes the packaged information. The satellite monitoring system transmits the analyzed information to a console deployed on a computing device at a command station. The satellite monitoring system provides communication between the console and the detection systems. The console receives the transmitted information from the satellite monitoring system and communicates user interaction based on the transmitted information to the satellite monitoring system.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and instrumentalities disclosed herein.

FIG. 1 illustrates the hazardous substance release notification system comprising a satellite monitoring system.

FIG. 2 exemplarily illustrates a satellite monitoring system of the hazardous substance release notification system.

FIG. 3 illustrates an embodiment of the hazardous substance release notification system comprising a radio frequency monitoring system.

FIG. 4 exemplarily illustrates a radio frequency monitoring system of the hazardous substance release notification system.

FIG. 5 illustrates the components of a console deployed at a command station.

FIG. 6 illustrates a method of obtaining information on the contaminants in ambient air.

FIG. 7 exemplarily illustrates the process flow involved in obtaining information on the contaminants using a satellite monitoring system.

FIG. 8 exemplarily illustrates the process flow involved in obtaining information on the contaminants using a satellite monitoring system and a radio frequency monitoring system.

FIG. 9 exemplarily illustrates simulation of display of information regarding the air quality as viewed by an operator at the command station.

FIG. 10 exemplarily illustrates the hazardous substance release notification system with sensors for obtaining information on contaminants in ambient air.

### DETAILED DESCRIPTION OF THE INVENTION

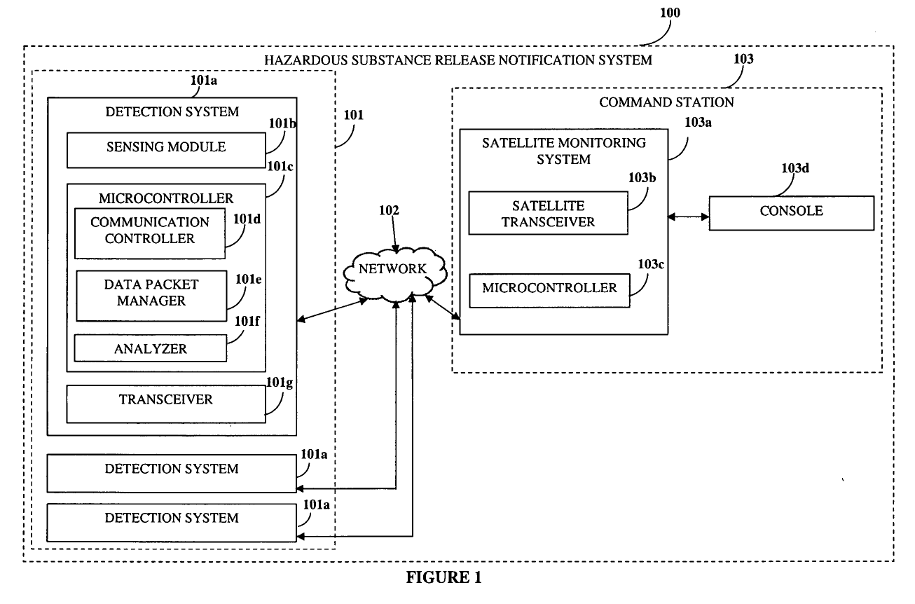

FIG. 1 illustrates the hazardous substance release notification system 100 (HSRNS) comprising a satellite monitoring system 103a. The system 100 disclosed herein comprises a plurality of detection systems 101, a satellite monitoring system 103a, and a console 103d. The satellite monitoring system 103a and the console 103d are deployed at a command station 103. The detection systems 101 communicate with the satellite monitoring system 103a via a network 102.

The detection systems 101 sample the ambient air for contaminants 701 in real time and publish information to the satellite monitoring system 103a. The contaminants 701 in the ambient air may be radiological contaminants, biological contaminants, chemical contaminants, and other airborne contaminants. The detection system 101a may be deployed in a mobile vehicle or a fixed location. As used herein, the term “vehicle” refers to any means of transportation or a container used for transportation of objects. The detection systems 101 may be installed in the vehicle equipped with a heating system, a ventilating system, and an air conditioning system. In the absence of the heating system, the ventilating system, and the air conditioning system in the vehicle, one or more fans may be directly integrated into the detection systems 101 or may be installed in the vehicle. The detection systems 101 suck air circulated by the fans.

Each of the detection systems 101 comprises a sensing module 101b, a microcontroller 101c, and a transceiver 101g. The sensing module 101b senses the contaminants 701 in the ambient air. The sensing module 101b may be a commercial off the shelf (COTS) component capable of sensing airborne contaminants 701 such as radiological contaminants, physical contaminants, and chemical contaminants. The sensing module 101b may use sensing components for obtaining information on contaminants 701 in ambient air. The sensing components comprise a Helium tube, a Lithium crystal, and a Geiger-Muller tube. The sensing module 101b may detect radiological contaminants, biological contaminants, and chemical contaminants. The radiological contaminants detected by the sensing module 101b comprise alpha, beta, gamma, and neutron radiation. The radiological contaminants detected by the sensing module 101b further comprise uranium and plutonium enriched compounds. The biological contaminants detected by the sensing module 101b comprise viruses and bacteria. The chemical contaminants detected by the sensing module 101b comprise organophosphorus compounds and lewisite compounds.

In addition to sensing contaminants 701, the sensing module 101b may also report findings in a variety of communication protocols. The microcontroller 101c comprises a communication controller 101d, a data packet manager 101e, and an analyzer 101f. The communication controller 101d provides a conduit for communication between the sensing module 101b and other sub systems of a detection system 101a. For outbound communications from the detection system 101a, the communication controller 101d may leverage one of the available transceivers, a satellite transceiver 103b, or a radio frequency transceiver 103j for completing a transmission. The communication controller 101d is an optional component since the transceiver 101g functionality may reside in the microcontroller 101c. Variants of the communication controller 101d may be deployed in one or more of the sub systems within the HSRNS 100. A single component may also perform the functions performed by the microcontroller 101c and the transceiver 101g.

In the implementation of the detection system 101a, the communication controller 101d may be resident on the microcontroller 101c hardware portion of the detection system 101a. Once the microcontroller 101c obtains the information on the contaminants 701 further analysis may be performed for determining hazardous substances in the contaminants 701. The analyzer 101f analyzes the contaminants 701 in the ambient air for hazardous substances. The communication controller 101d may be leveraged again for the transmission of the analyzed information. The recipient of the enhanced information transmission may either be the detector system 101a, the radio frequency monitoring system 103i or the satellite monitoring system 103a. The communication protocols may be one of a wireless communication protocols such as Bluetooth™, 802.11, radio frequency identification (RFID) protocol, satellite 702, infrared, or wired such as digital and analog signals for serial communication. For outbound communications from the detection system 101a either the satellite transceiver 103b or the radio frequency transceiver 103j may be used.

The transceiver 101g transmits information on the hazardous substances to the satellite monitoring system 103a. The transceiver 101g may have a COTS component allowing communications into and out of the detection system 101a. The transceiver 101g on the detection system 101a may be a satellite transceiver 103b or radio frequency transceiver 103j. For example, for direct satellite communication, a satellite transceiver 103b may be used for transmitting the information on the hazardous substances to the satellite monitoring system 103a at the command station 103. In case direct satellite communication is infeasible, a radio frequency transceiver 103j may be used for transmitting the information on the hazardous substances to a radio frequency monitoring system 103i at the command station 103. The transceiver 101g transmits the information on the hazardous substances as data packets to the satellite monitoring system 103a. The data packet manager 101e parses, constructs, and defines data packets according to a predefined transmission protocol. In addition, the transceiver 101g may provide standard cable communication capabilities such as RJ45, RJ11, serial, parallel, digital, and universal serial bus. The transceiver 101g may also allow information to be communicated back into the detection systems 101.

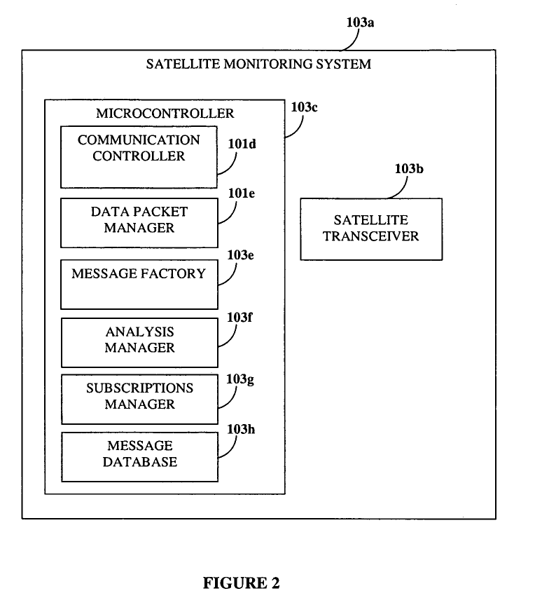

The satellite monitoring system 103a comprises a satellite transceiver 103b and a microcontroller 103c as illustrated in FIG. 2. The satellite transceiver 103b receives the data packets from the detection systems 101. The microcontroller 103c of the satellite monitoring system 103a packages the information transmitted from each of the detection systems 101 and analyzes the packaged information. The microcontroller 103c therefore prepares the analyzed information and communicates the analyzed information to the satellite transceiver 103b for transmission to the console 103d. The satellite transceiver 103b may be a COTS component capable of broadcasting and receiving satellite signals. The satellite transceiver 103b then transmits the analyzed information via a satellite signal to the console 103d deployed on a computing device 703 at the command station 103. The computing device 703 may be an operator's computer.

The microcontroller 103c of the satellite monitoring system 103a comprises a communication controller 101d, a data packet manager 101e, a message factory 103e, and analysis manager 103f, a subscriptions manager 103g, and a message database 103h as illustrated in FIG. 2. The communication controller 101d may act as a gate keeper for data transmission between the detection system 101a and the satellite monitoring system 103a. The communication controller 101d may also be the entry point for the satellite monitoring system 103a once the satellite transceiver 103b receives the data packets. The communication controller 101d may coordinate conversion and formatting of the data packets using the data packet manager 101e.

While the communication controller 101d is the gate keeper of intra and external communication, the message factory 103e is a software component that constructs and brokers the information contained in the communication. The message factory 103e constructs the message comprising the analyzed information. When the message factory 103e completes the construction of the message and publishes the message, the subscriptions manager 103g is alerted.

The message factory 103e parses data in the analyzed information for ensuring validity and identification of a “message header” type. Based on the header type, the message factory 103e attempts to locate the appropriate “message body” and constructs a complete message populated with the information received from the data transmission. Exemplarily, the resultant message is a Plain Old Java Object “POJO”. The message may be published to a queue in a message database 103h where the subscriptions manager 103g processes the message. All activity and events resulting in core behavior, identity, deletion, and updation of on screen objects need to follow the messaging schema. For cosmetic and display variation changes, the display may be achieved by directly manipulating the objects properties.

The analysis manager 103f acts as an entry point for request for statistical information. Core functionality of the analysis manager 103f provides “hot spot” identification, real time positioning of mobile sensors, and contamination level readings of each of the mobile sensors. The information available from the analysis manager 103f may be used collaboratively with third party information sources such as ARC internet map server (ARC IMS). ARC is a lossless data compression and archival format. The ARC IMS delivers map information, produced by environmental systems research institute (ESRI) and geographic information system (GIS), across the internet. Further depth may be applied to the mobile sensors findings by applying weather data. The analysis manager 103f may also provide insight into training scenarios, areas of improvement for first responders, response times, and predictability studies.

The mobile sensors may participate in the detection system 101a via a subscriptions manager 103g. On start up of each of the deployed mobile sensor, the mobile sensors may automatically transmit a signal for registering the mobile sensor with a transceiver 101g. The registration of the mobile sensor with a transceiver 101g defines the topics and types of information to be transmitted to the mobile sensor by the transceiver 101g. Once the communication channel between the mobile sensor and the transceiver 101g is established, the communication channel may be used for performing activities comprising transmitting commands, performing updates, performing diagnostics, performing data upload, and performing data download.

The satellite monitoring system 103a therefore aggregates information transmitted from the detection systems 101, publishes the aggregated information, and relays the published information to the console 103d for presentation to an operator's workstation. The satellite monitoring system 103a may physically be located at the command station 103 with a connection to the operator's workstation. The operator's workstation may be used to display the received information on the console 103d. The satellite monitoring system 103a may also comprise software implemented in the microcontroller 103c to perform a more detailed analysis relevant to the aggregate level information. In addition, the satellite monitoring system 103a may have the intelligence and authority to modify or flag situations that may require attention.

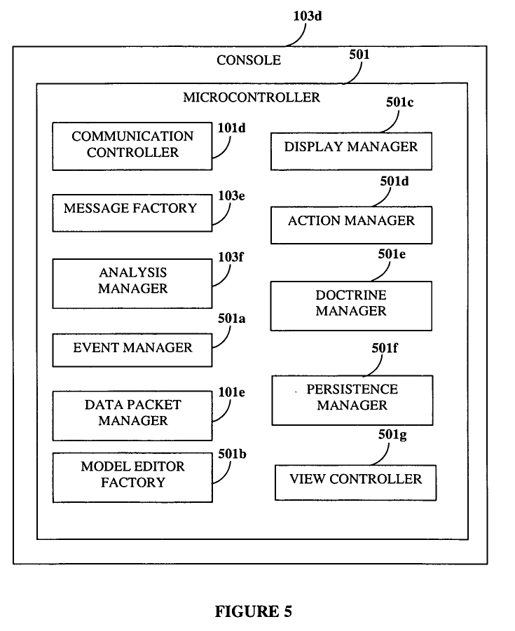

The console 103d may be deployed on a computer at the command station 103. The console 103d receives information from the satellite monitoring system 103a, communicates user interaction back to the satellite monitoring system 103a and prepares the information for displaying the information to an operator such as a law enforcement official at the command station 103. The components of the console 103d deployed at the command station 103 are illustrated in FIG. 5. The console 103d may comprise a microcontroller 501. The microcontroller 501 of the console 103d comprises a communication controller 101d, a data packet manager 101e, a message factory 103e, an analysis manager 103f, an event manager 501a, a model editor factory 501b, a display manager 501c, an action manager 501d, a doctrine manager 501e, a persistence manager 501f, and a view controller 501g.

The console 103d may be installed in the operator's workstation in the command station 103. The console 103d may render a graphical representation of the analyzed information transmitted by the satellite monitoring system 103a. The console 103d further enables user interaction and communication with other subsystems via the satellite monitoring system 103a connected to the workstation. The communication controller 101d, the data packet manager 101e, the message factory 103e, and the analysis manager 103f are explained in the detailed description of FIG. 1.

The doctrine manager 501e facilitates calibration of the system alarms and response protocols in the event of sensing contaminants 701. The rules applied may range from moderate to severe. A moderate rule may be to log information and flag a vehicle as a potential threat. A severe rule may be to send a message inhibiting engine functions of the vehicle and alerting law enforcement. The doctrine manager 501e may define thresholds, response protocols, communication protocols, and plans of action, filtering, and escalation priorities. Each of the properties of the doctrine manager 501e may be configured independently according to the situation and contaminant and may execute the property with or without human intervention.

The persistence manager 501f stores objects and related information into a non volatile data repository. Non volatile data repositories may exemplarily be file systems and relational databases. Additional functions of the persistence manager 501f may be serialization and de-serialization of objects. Persistence functionality is an integral feature for supporting doctrine, reporting, and analysis features.

The display manager 501c allows the operator to interact with various functions of the HSRNS 100. The display at the console 103d is the operator's interface with the HSRNS 100. In addition to rendering graphical images on the screen of the console 103d, the display manager 501c may handle more complex functions including interpreting data models into graphics, screen dimensioning functions, relational mapping between graphics, and rendering optimization.

The display manager 501c may also comprise a display factory for dynamically matching data models to graphical representations at runtime. The display manager 501c may also control skinning and custom symbology for supporting military and industry standards.

In scenarios where a third party display is used such as falcon view, national aeronautics and space administration (NASA's), world wind, or Google™ Earth of Google™ Inc., the display manager 501c may expose an application programming interface (API) to expose the data models to the display. FalconView is a Windows® mapping system of Microsoft® Inc. displaying various types of maps and geographically referenced overlays.

The action manager 501d handles actions generated as a result of user interaction. Every mouse click or keyboard short cut may result in an action handled by the action manager 501d. The result of actions that are handled by the action manager 501d may translate into a message to the system that affects the identity, behavior, or cosmetic properties of the data model.

In situations where cosmetic or display type changes are required, the action manager 501d may allow direct editing of the properties of the object. In order to enable an operator to edit the data model, the operator uses a user interface also known as an editor on the console 103d. The instructions and sequences of events pairing a data model to an editor may be a function of the model editor factory 501b. The model editor factory 501b may be coupled with the action manager 501d since the model editor factory 501b receives instructions from the action manager 501d. Other functions of the action manager 501d are object cloning, serialization and change commit.

The event manager 501a is a software component similar in function to the action manager 501d. The event manager 501a responds to some action generated by the operator. In addition to the operator generated events, processes in other sub-systems may also generate events. Examples of events are additions of objects to queues and message publications.

The view controller 501g optimizes the rendering and display of graphical objects. The display may be visualized as a series of layers with each layer having a priority associated with it. Non volatile data such as background terrain information would be rendered at the deepest layers. For the top most layers where most user interaction may be transpired, graphical representations of the mobile sensors may be rendered. The view controller 501g may determine data to be drawn, location of drawing the data, time of drawing of the data, and frequency of refreshing display of the data.

The event manager 501a residing on the console 103d may record events generated in the console 103d. The events generated in the console 103d may either be system generated or user generated. The user generated events may be generated by interacting with the user interface.

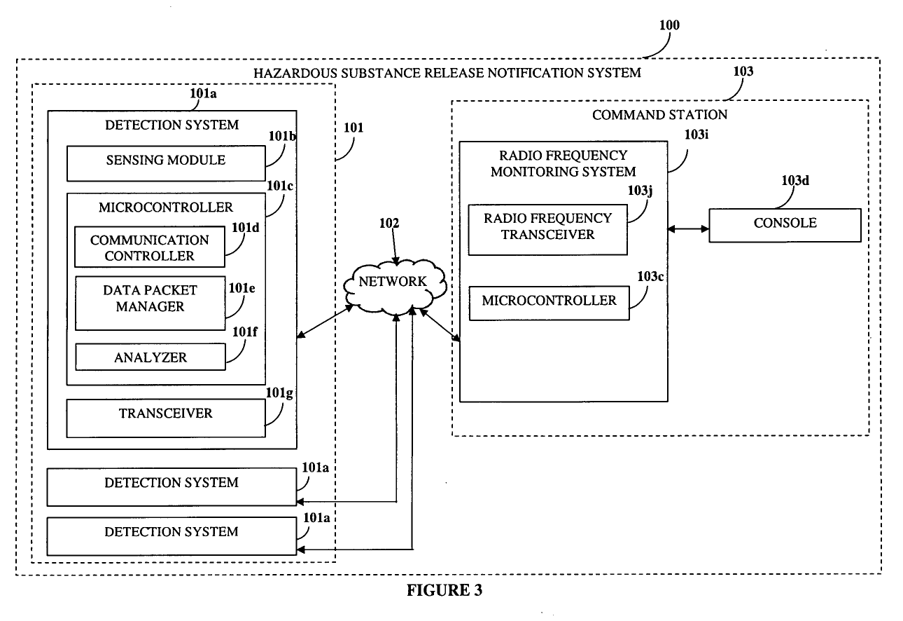

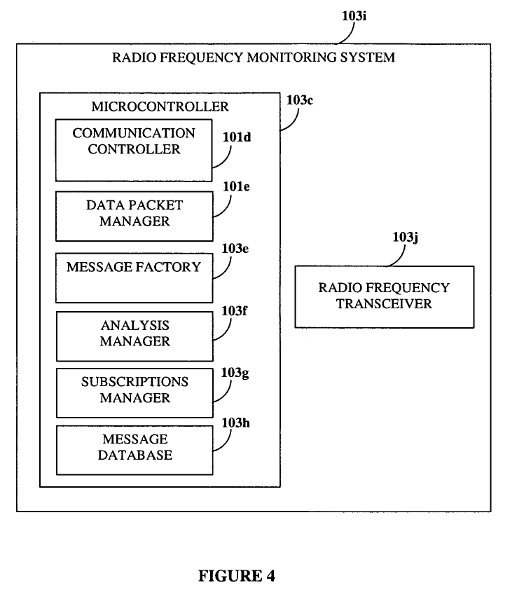

FIG. 3 illustrates an embodiment of the hazardous substance release notification system 100 comprising a radio frequency monitoring system 103i. The radio frequency monitoring system 103i is deployed at predefined transmission points and exemplarily may be used in situations where wireless fidelity technology (WiFi) is preferred. The radio frequency monitoring system 103i may be used on infeasibility of direct satellite communication. The infeasibility of direct satellite communication may exemplarily occur in situations where the detection system 101a does not comprise a satellite transceiver 101g. The radio frequency monitoring system 103i is exemplarily illustrated in FIG. 4. The radio frequency monitoring system 103i comprises a microcontroller 103c and a radio frequency transceiver 103j. The functions of the microcontroller 103c are explained in the detailed description of FIG. 1. The radio frequency transceiver 103j receives the data packets from the detection systems 101 and transmits the analyzed information of the hazardous substances to the console 103d. The radio frequency monitoring system 103i enables communication between the operator or console 103d and the detection system 101a. The difference between the radio frequency monitoring system 103i and the satellite monitoring system 103a is the radio frequency functionality found in the radio frequency monitoring system 103i. In situations where satellite communication is infeasible, the radio frequency monitoring system 103i would be the choice. For example, the radio frequency monitoring system 103i may be chosen over the satellite monitoring system 103a as a communication mode in subway tunnels, buildings, and deployments where there is a space requirement for the detection system 101a.

In a typical deployment of the radio frequency monitoring system 103i, one or more radio frequency monitoring systems may be deployed at various checkpoints in a field to communicate with mobile sensors in the detection system 101a. An example of this scenario in practice is vehicles with E-PASS transponders that communicate with receivers at every entry and exit point on a highway.

The radio frequency transceiver 103j may be a COTS component capable of sending and receiving radio frequencies to wireless devices. The radio frequency transceiver 103j may also communicate between the detection system 101a and the satellite transceiver 103b delivering information to and from the operator. Examples of protocols supporting communication between the detection system 101a and the radio frequency transceiver 103j are IEEE 802.11, RFID protocol, infrared, analog and digital.

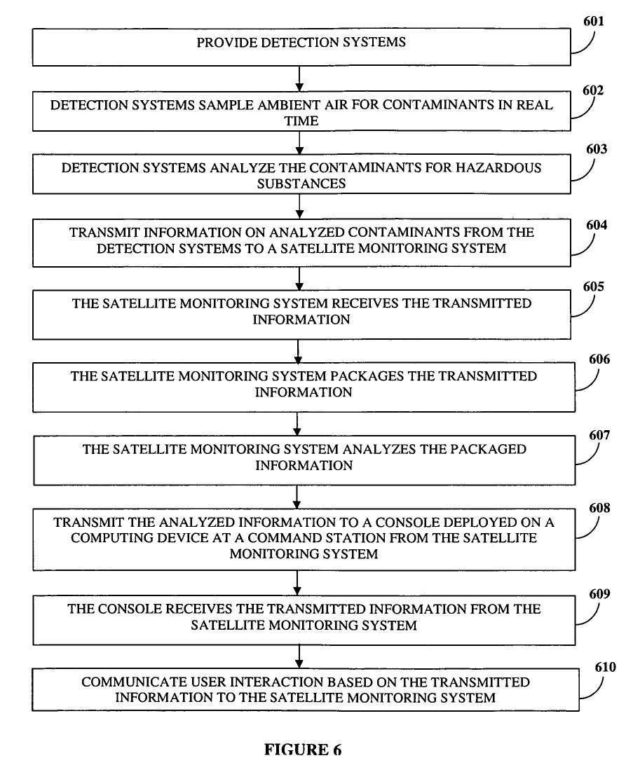

FIG. 6 exemplarily illustrates a method of obtaining information on the contaminants 701 in ambient air. The method disclosed herein provides 601a plurality of detection systems 101. The detection systems 101 may be deployed in a vehicle or at a fixed location. The detection systems 101 sample 602 the ambient air for contaminants 701 in real time. The detection systems 101 then analyze 603 the contaminants 701 for hazardous substances. Each of the detection systems 101 may package information on contaminants 701, vehicle information, and global positioning system information. The information packaging is performed on one of triggering an alarm and on reaching a predefined threshold. The detection systems 101 transmit 604 information gathered on the hazardous substances to a satellite monitoring system 103a as data packets.

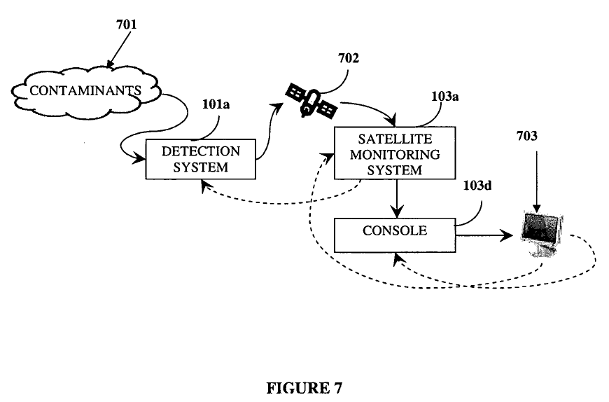

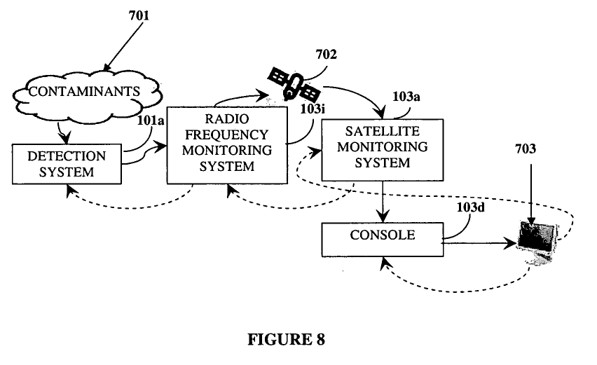

The satellite monitoring system 103a receives 605 the transmitted information from each of the detection systems 101. The satellite monitoring system 103a packages 606 the transmitted information received from each of the detection systems 101. The satellite monitoring system 103a then analyzes 607 the packaged information. The satellite monitoring system 103a transmits 608 the analyzed information to a console 103d deployed on a computing device 703 at the command station 103 via a network 102. The console 103d receives 609 the transmitted information from the satellite monitoring system 103a. The console 103d communicates 610 the user interaction to the satellite monitoring system 103a, based on the transmitted information. Radio frequency identification tags provided in each of the detection systems 101 may detect tampering of the detection systems 101. The console 103d may also graphically represent the transmitted information from each of the detection systems 101. The transmitted information based on the analyzed contaminants comprising hazardous substances is thereby notified to the command station 103. The process flow involved in obtaining information on the contaminants 701 using the satellite monitoring system 103a is exemplarily illustrated in FIG. 7. The process flow involved in obtaining information on the contaminants 701 using the satellite monitoring system 103a and the radio frequency monitoring system 103i is exemplarily illustrated in FIG. 8.

Consider an example of the method and system disclosed herein. As used herein, the term “receiver unit” refers to the radio frequency monitoring system 103i or the satellite monitoring system 103a depending on the implementation. A vehicle with a portable device continuously samples the ambient air for hazardous elements and then broadcasts a wireless signal to a tower if a positive reading is encountered. The wireless signal may comprise a chemical finding, level of contamination and position of the vehicle using existing global positioning system (GPS) technology. A receiver unit consumes the wireless signal and takes action according to its mandate. The receiver unit may be placed in and around areas of interest and may be used to setup a perimeter to identify mobile threats. As used herein, the presence of chemical, biological, nuclear, or illegal drugs intended for malicious activities will be referred to as a “threat”. The method and system disclosed herein may exemplarily be used in monitoring and notifying release of hazardous substances in a tunnel used for pedestrian and commercial traffic, proactively monitoring a vehicle that is a potential threat to the safety of the citizens in and around a tunnel, etc.

In case the vehicle is equipped with a heating system, a ventilating system, and an air conditioning system, the detection systems 101 are installed. In case the vehicle is not equipped with the heating system, the ventilating system, and the air conditioning system fans are installed in the vehicle. The detection systems 101 suck the air circulated by the fans. Each of the detection systems 101 packages information. The packaged information comprises information on contaminants 701, vehicle information, and global positioning system information. The information packaging may be performed on triggering an alarm or on reaching a predefined threshold. For example, the information packaging may be performed in case a detection system 101a detects a hazardous substance that may be critical for survival of humans.

The satellite monitoring system 103a aggregates the information from the detection systems 101. The satellite monitoring system 103a provides communication between a console 103d and the detection systems 101. The satellite monitoring system 103a receives the transmitted information. The satellite monitoring system 103a packages the transmitted information and analyzes the packaged information. The satellite monitoring system 103a transmits the analyzed information to the console 103d deployed on a computing device 703 at a command station 103. The console 103d receives the transmitted information from the satellite monitoring system 103a and communicates user interaction based on the transmitted information to the satellite monitoring system 103a. The console 103d graphically represents the transmitted information from each of the detection systems 101. A radio frequency monitoring system 103i may be used for receiving and transmitting information on the analyzed contaminants 701. The radio frequency monitoring system 103i may be used in case direct satellite communication is infeasible.

The detection system 101a that is resident on the vehicle comprises a plurality of radio frequency identification (RFID) tags. The detection system 101a analyzes the ambient air for hazardous substances and notifies the receiver unit of the information on the analyzed ambient air. The RFID tags check on the proper functioning of the sensor network on the vehicle.

The following example illustrates an application of the HSRNS 100. Consider a tunnel used for pedestrian and commercial traffic. There is a potential threat of unleashing of hazardous materials in or around the tunnel or even detonating of an explosive device. The challenge is to efficiently and proactively monitor each vehicle that may be a potential threat to the safety of citizens in and around the tunnel. The operator of the vehicle or the contents of the vehicle may release vapors that may be detected and evaluated for hazardous materials in real time by a unit on or in the vehicle. The receiver units are installed in a half mile radius of the tunnel and constantly wait for a positive signal from vehicles passing by. Once a vehicle transmits a positive signal, the information is transferred to appropriate authorities. The authorities may track the vehicle using GPS or visual methods. Therefore the authorities may be given ample distance and time to avert a potential disaster.

While the proposed initial implementation of the HSRNS 100 addresses the above mentioned scenario, there are other situations or venues that lend themselves to this HSRNS 100. While this scenario describes a mobile threat and fixed target area, it is conceivable that such a HSRNS 100 may be deployed in airports, office buildings, subways. Another embodiment of the HSRNS 100 may comprise a portable receiver that may be implemented in existing structures and have the detection system 101a of the HSRNS 100 remain the same. An example of where this embodiment of the HSRNS 100 may be employed are shipping ports with containers outfitted with the detection system 101a of the HSRNS 100 and with portable receiver units deployed at various points of the shipping port.

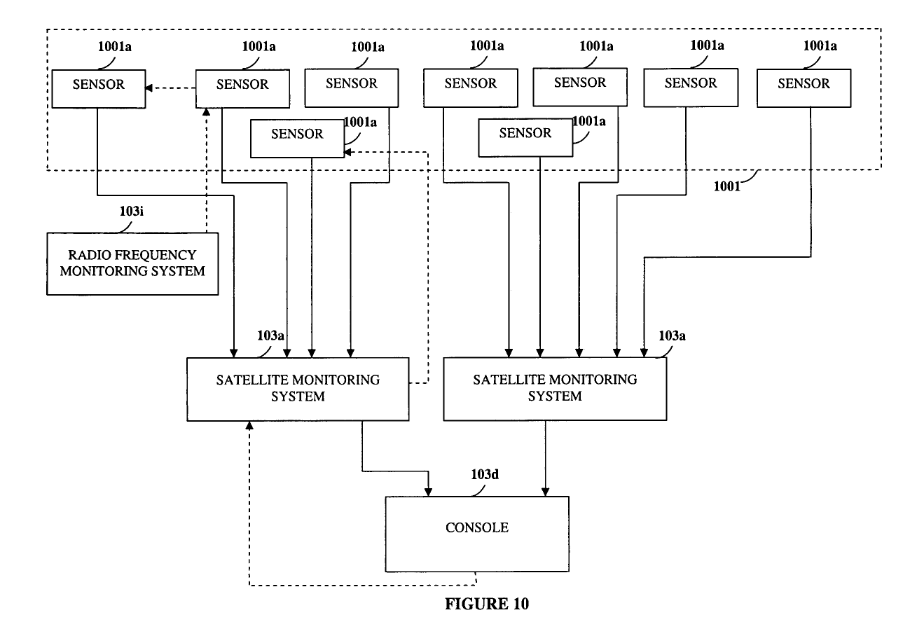

The detection systems 101 may include a plurality of sensors 1001 as illustrated in FIG. 10. The detection system 101a comprises a sensor 1001a, capable of analyzing ambient air and airborne matter and for detecting hazardous substances. Ambient air is the air in the vicinity of one or more of the sensing units, or the air that is drawn past the sensor 1001a. Each sensor 1001a is a combination of hardware and software. In the case where the sensor 1001a is an RFID tag, there may be a software component resident in the RFID tag. A communication controller software is resident on the sensor 1001a. The sensor 1001a may communicate with one or more sensors 1001 and multiple RFID tags for providing an accurate assessment of the ambient air and airborne matter. The sensor 1001a may comprise a global positioning system (GPS) functionality to provide the vehicle position in real time.

The sensors 1001 may be tied directly into the vehicles heating, ventilating, and air conditioning (HVAC) system or may be a stand alone sensing unit in the vehicles cabin. A sensor 1001a may exemplarily monitor HVAC systems including the HVAC system in planes, trains, boats, cars, etc. The stand alone sensing units may be employed in shipping containers. If the vehicle is equipped with a HVAC system, the detection system 101a may be installed in parallel and fed return air. If a HVAC system is not present in the detection system 101a, fans may be installed in the vehicle and the detection system 101a may solely rely on suction of the air circulated by the fan. Depending on the requirements of the implementation, the fans may be incorporated and may be calibrated specifically for the implementation. Specifications relating to the displacement of air and the volume of the space may be taken into consideration during selection of a fan or an air circulation method. The integrating the fan into the detection system 101a may be applicable for warehouses, storage facilities, and shipping containers.

Integration of RFID tags into the detection system 101a and the immediate surrounding environment of the detection system 101a may be used as an anti tampering system. The function of the RFID tags may be to validate the proper functioning of the detection system 101a and act as tamper deterrent. Each of the RFID tags may transmit information such as the proper functioning of the detection system 101a to a receiver unit. A plurality of RFID tags is located at various points in the vehicle. The RFID tags are both passive and active. The RFID tags communicate with each other as well as the sensors 1001 via the controller software residing on each of the sensors 1001. The ability of the RFID tags to transmit information directly to the receiver unit may depend on the type of implementation of the RFID tags. The RFID tags may be used for gathering information as to functioning status and availability of other participating RFID tags. The RFID tags may be used for gathering information as to functionality and availability of the sensors 1001.

The microcontroller 103c may be a software brokering communication and activities between other software processes. Each sensor 1001a may comprise a proprietary piece of software responsible for general communication within the system as well as communication with external receivers. The proprietary piece of microcontroller 103c software may be a controller for a unit the proprietary piece of controller software is deployed on. The detection system 101a may comprise a microcontroller 101c responsible for functions of the sensor network and provide an interface as an entry point for external components and systems. The receiver unit may also comprise a microcontroller 103c responsible for brokering processes on the receiver unit as well as for providing an entry point for external components and systems.

The microcontroller 103c may comprise features configurable via a user interface and may comprise driver level code to communicate with hardware. The detection system 101a may be capable of asynchronous communication between the local network comprising RFID and other sensors 1001 and the receiver via the software resident on the detection system 101a. The software may be capable of communication with the necessary hardware components of the detection system 101a as well as adhere to the required transmission protocol. The microcontroller 103c may also comprise functionalities for performing logic and maintenance as needed. A variation of the microcontroller 101c and 103c may be developed for deployment on the both the receiver and sensor components.

Each receiver unit may comprise a hardware and software element. The microcontroller 103c of the receiver unit may function as the microcontroller 101c on the detection system 101a with an additional functionality of providing communication to a third party system. For example, the microcontroller 103c of the receiver unit may send a notification to a person, system, or a group via wireless, satellite 702, email, voice, network protocols, etc. The analytical component of the receiver unit such as the analysis manager 103f identifies trends, patterns, and false positives. For example, the analytical component of the receiver unit may identify contamination in the atmosphere.

A fixed detection system 101a comprising a detection unit and RFID tags may be located in a mobile entity. For example, the fixed detection system 101a comprising a detection unit and RFID tags may be located in an automobile, subway car, shipping container, etc. The detection unit may be in constant communication with the RFID tags located in the vehicle. Each of the participating RFID tags may be lesser in intelligence to the actual detection unit and may not comprise analysis capabilities for monitoring the air. In some cases the RFID tags may be passive and simply exist for sanity checks for the sensor network. In another embodiment the RFID tags may have intelligence to communicate finding from the detection system 101a to an external receiver. If the sensing module 101b of the detection system 101a fails a sanity check with the RFID tags, or encounters an abnormal reading of the air quality, the RFID tags may transmit findings to an external receiver unit. In case the sensing module 101b functions as desired, the RFID tags may sample ambient air and airborne particles, for example compartment air in a vehicle, in real time.

The sampled air may be analyzed in real time by the communication controller 101d software on the detection system 101a for hazardous substances. If analysis of the air sampled is positive for a hazardous substance, the microcontroller 101c in the detection system 101a and possibly additional software associated with the active RFID tags, assembles the information on the detection system 101a, fixes the location of the detection system 101a and location of the vehicle using GPS technology, encrypts the detection system 101a information with any other necessary information, packages the message into multiple or specific wireless formats, and transmits the detection system 101a information to the receiver unit programmed to receive such transmission. In case the receiver unit is within range, the receiver unit receives and decrypts the message, identifies the hazardous substance and priority level using proprietary software on the receiver unit, and transmits a notification of the hazardous substance detected, location of hazardous substance and the priority level to the police or regulatory authority for appropriate action. As a result of consuming the detection system 101a information, the regulatory authority may take appropriate course of action for the situation. Such action may include notification of law enforcement officials, set road blocks, quarantine vehicle or location or communicate back to the individual detection system 101a.

The software on the receiver unit may enforce business logic and communication protocols. Depending on the requirements of the organization deploying the HSRNS 100 there may be a need to capture and analyze data before any alarms are triggered or special rules apply. Also, the method and rules of data transmission are resident on the receiver unit and the method and rules of data transmission define the transmission protocols of the organization. The software on the receiver unit identifies dispatch information according to the communication protocols that were defined by the organization that deployed the receiver unit. The recipients of the transmission may be an individual, organization, the police, or regulatory authorities. While the communication protocol is defined by the organization deploying the detection system 101a the default implementation may be a method of wireless communication. Other possible modes of communication may exemplarily include voice, e-mail, fax, satellite audio or visual, etc.

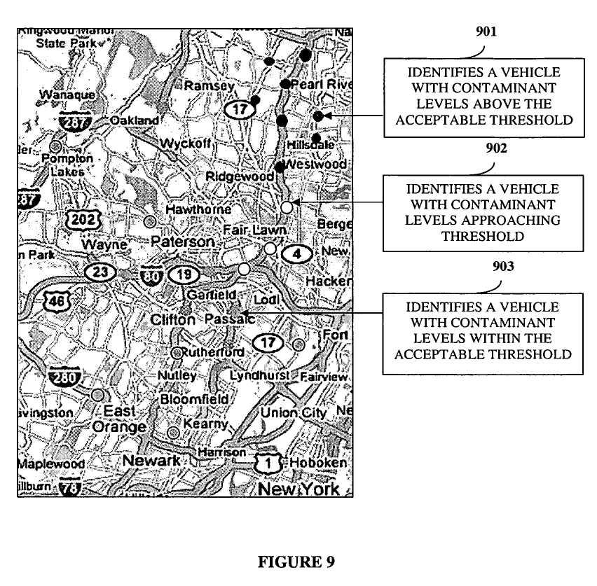

FIG. 9 illustrates a simulation of the display of information regarding the air quality as viewed by an operator at the command station 103. The console 103d may be manned by a law enforcement official. The console 103d graphically represents the information transmitted from each of the detection systems 101. The information may be presented over a map to provide a geographic depiction. The console 103d may also provide a feature rich suite of tools to assist the operator in making decisions and taking action. The simulation of the sensor data on an overlay over a map of a geographic area and may be depicted over a satellite image of the geographic area if required. The depiction of the geographic area may not demonstrate the full capability of the system and is only intended to give a simple visual aid to conceptualize the display. The solid black circles may identify 901 contaminant vehicles above the acceptable threshold. The white circles may identify 902 contaminant vehicles approaching the acceptable threshold. The gray circles may identify 903 contaminant vehicles within the acceptable threshold.

In one embodiment of the method and system disclosed herein, there are many mobile transmitters at any given geographic location. A typical scenario may be vehicles traveling on a highway and transmitting information regarding the air quality of the current location and the air quality of the location the vehicle is heading. While the content of the vehicle may be clear of contaminants 701 the atmosphere through which the vehicle travels may contain contaminants 701 as a result of intentional or accidental release of hazardous substances into the atmosphere. A central monitoring station collects the information on the contaminants 701 in the atmosphere from all of the available information from the vehicle and provides a visual representation of the collected information. In theory the information would define a contamination boundary and real time tracking of the contaminations movement. The information may be further enhanced with the addition of information collected from other vehicles such as planes, trains, shipping containers, etc. Once the additional information is combined with the existing sampling the result may yield a three dimensional picture from sub-surface, surface, above sea level and diameter of the effected zone. The information may assist early response units and authorities of safe zones and may play a critical role in evacuation and containment of the hazardous substances.

Consider another example of the HSRNS 100 with multiple detection systems 101 with sensors 1001 for obtaining information on the contaminants 701 in the ambient air as illustrated in FIG. 10. The sensors 1001 report their findings on the contaminants 701 in the ambient air to the nearest receiver unit. The receiver unit may be the satellite monitoring system 103a or the radio frequency monitoring system 103i. The receiver unit performs analysis on the information of the contaminants 701 and transmits the analyzed information to the console 103d. The console 103d receives the transmitted information from the receiver unit and may send commands to one or more sensors 1001 via the receiver unit. The receiver unit may communicate with the sensors 1001 without the permission of the console 103d. The console 103d may communicate with each individual sensor 1001a or may have the sensor 1001a to console 103d communication brokered by the receiver unit. A hardwired hub or a radio frequency monitoring system 103i may be used for deployment where direct access to the satellite monitoring system 103a is not available.

It will be readily apparent that the various methods and algorithms described herein may be implemented in a computer readable medium, e.g., appropriately programmed for general purpose computers and computing devices. Typically a processor, for e.g., one or more microprocessors will receive instructions from a memory or like device, and execute those instructions, thereby performing one or more processes defined by those instructions. Further, programs that implement such methods and algorithms may be stored and transmitted using a variety of media, for e.g., computer readable media in a number of manners. In one embodiment, hard-wired circuitry or custom hardware may be used in place of, or in combination with, software instructions for implementation of the processes of various embodiments. Thus, embodiments are not limited to any specific combination of hardware and software. A “processor” means any one or more microprocessors, Central Processing Unit (CPU) devices, computing devices, microcontrollers, digital signal processors, or like devices. The term “computer-readable medium” refers to any medium that participates in providing data, for example instructions that may be read by a computer, a processor or a like device. Such a medium may take many forms, including but not limited to, non-volatile media, volatile media, and transmission media. Non-volatile media include, for example, optical or magnetic disks and other persistent memory volatile media include Dynamic Random Access Memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during Radio Frequency (RF) and Infrared (IR) data communications. Common forms of computer-readable media include, for example, a floppy disk, a flexible disk, hard disk, magnetic tape, any other magnetic medium, a Compact Disc-Read Only Memory (CD-ROM), Digital Versatile Disc (DVD), any other optical medium, punch cards, paper tape, any other physical medium with patterns of holes, a Random Access Memory (RAM), a Programmable Read Only Memory (PROM), an Erasable Programmable Read Only Memory (EPROM), an Electrically Erasable Programmable Read Only Memory (EEPROM), a flash memory, any other memory chip or cartridge, a carrier wave as described hereinafter, or any other medium from which a computer can read. In general, the computer-readable programs may be implemented in any programming language. Some examples of languages that can be used include C, C++, C#, or JAVA. The software programs may be stored on or in one or more mediums as an object code. A computer program product comprising computer executable instructions embodied in a computer-readable medium comprises computer parsable codes for the implementation of the processes of various embodiments.

Where databases are described such as the message database 103h, it will be understood by one of ordinary skill in the art that (i) alternative database structures to those described may be readily employed, and (ii) other memory structures besides databases may be readily employed. Any illustrations or descriptions of any sample databases presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats including relational databases, object-based models and/or distributed databases could be used to store and manipulate the data types described herein. Likewise, object methods or behaviors of a database can be used to implement various processes, such as the described herein. In addition, the databases may, in a known manner, be stored locally or remotely from a device that accesses data in such a database.

The present invention can be configured to work in a network environment including a computer that is in communication, via a communications network, with one or more devices. The computer may communicate with the devices directly or indirectly, via a wired or wireless medium such as the Internet, Local Area Network (LAN), Wide Area Network (WAN) or Ethernet, Token Ring, or via any appropriate communications means or combination of communications means. Each of the devices may comprise computers, such as those based on the Intel® processors, AMD® processors, Sun® processors, IBM® processors etc., that are adapted to communicate with the computer. Any number and type of machines may be in communication with the computer.

The foregoing examples have been provided merely for the purpose of explanation and are in no way to be construed as limiting of the present method and system disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may effect numerous modifications thereto and changes may be made without departing from the scope and spirit of the invention in its aspects.

I claim:

1. A system for obtaining information on contaminants in ambient air, comprising:
a plurality of detection systems for sampling said ambient air for said contaminants in real time, wherein each of said detection systems comprises:
a first microcontroller comprising an analyzer for analyzing the contaminants for hazardous substances; and
a first transceiver for transmitting information on said hazardous substances to a satellite monitoring system, wherein said information on the hazardous substances is transmitted as data packets;
said satellite monitoring system comprising:
a second microcontroller for packaging the information transmitted from each of the detection systems and analyzing said packaged information;
a second transceiver for transmitting said analyzed information to a console deployed on a computing device at a command station; and
said console for receiving said transmitted information from the satellite monitoring system and communicating user interaction based on the transmitted information to the satellite monitoring system.

2. The system of claim 1, wherein each of the detection systems comprises a sensing module for sensing the contaminants in the ambient air.

3. The system of claim 1, wherein the satellite monitoring system provides communication between the console and the detection systems, further wherein the satellite monitoring system aggregates the information from the detection systems.

4. The system of claim 1, wherein said second transceiver of the satellite monitoring system receives said data packets transmitted by each of the detection systems.

5. The system of claim 1, wherein one or more of the detection systems is deployed in a vehicle.

6. The system of claim 5, wherein the detection systems are installed in said vehicle equipped with a heating system, a ventilating system, and an air conditioning system.

7. The system of claim 5, wherein one or more fans are installed on the detection systems in absence of a heating system, a ventilating system, and an air conditioning system in said vehicle, wherein the detection systems suck air circulated by said fans.

8. The system of claim 1, further comprising a radio frequency monitoring system for receiving and transmitting information on the hazardous substances from the detection systems on infeasibility of direct satellite communication.

9. The system of claim 1, wherein one or more of the detection systems is deployed at a fixed location.

10. The system of claim 1, wherein one or more of the detection systems comprises radio frequency identification tags for detecting tampering of the detection systems.

11. The system of claim 1, wherein the console graphically represents the transmitted information from the satellite monitoring system.

12. The system of claim 1, wherein said first microcontroller packages information on contaminants, vehicle information, and global positioning system information, wherein said information packaging is performed on one of triggering an alarm and on reaching a predefined threshold.

13. A method of obtaining information on contaminants in ambient air, comprising the steps of:
providing a plurality of detection systems for performing the steps of:
sampling said ambient air for said contaminants in real time;
analyzing the contaminants for hazardous substances;
transmitting information on said hazardous substances to a satellite monitoring system, wherein said information on the hazardous substances is transmitted as data packets;
receiving said transmitted information by said satellite monitoring system from each of said detection systems, wherein the satellite monitoring system packages the transmitted information, further wherein the satellite monitoring system analyzes said packaged information;
transmitting said analyzed information to a console deployed on a computing device at a command station by the satellite monitoring system; and
receiving said transmitted information by said console from the satellite monitoring system and communicating user interaction based on the transmitted information to the satellite monitoring system;
whereby the transmitted information based on said analyzed contaminants comprising hazardous substances is notified to said command station.

14. The method of claim 13, further comprising a step of providing communication between the console and the detection systems by the satellite monitoring system, wherein the satellite monitoring system aggregates the information from the detection systems.

15. The method of claim 13, wherein one or more of the detection systems is deployed in a vehicle.

16. The method of claim 13, wherein one or more of the detection systems is deployed at a fixed location.

17. The method of claim 13, further comprising a step of detecting tampering of the detection systems by radio frequency identification tags in the detection systems.

18. The method of claim 13, further comprising a step of graphically representing the transmitted information from each of the detection systems on the console.

19. The method of claim 13, further comprising a step of packaging the information on the contaminants, vehicle information, and global positioning system information by each of the detection systems, wherein said information packaging is performed on one of triggering an alarm and on reaching a predefined threshold.

20. The method of claim 13, wherein the contaminants are one or more of radiological contaminants, biological contaminants, and chemical contaminants, further wherein the contaminants are airborne contaminants.

21. A computer program product comprising computer executable instructions embodied in a computer readable medium, wherein said computer program product comprises:
a first computer parsable program code for analyzing contaminants for hazardous substances by a plurality of detection systems;
a second computer parsable program code for transmitting information on said hazardous substances to a satellite monitoring system from said detection systems;
a third computer parsable program code for receiving said transmitted information by said satellite monitoring system from each of the detection systems;
a fourth computer parsable program code for packaging and analyzing the transmitted information by the satellite monitoring system; and
a fifth computer parsable program code for transmitting said analyzed information to a console deployed on a computing device at a command station by the satellite monitoring system.

22. The computer program product of claim 21, further comprising a sixth computer parsable program code for graphically representing the transmitted information from each of the detection systems.

23. The computer program product of claim 21, further comprising a seventh computer parsable program code for packaging information on the contaminants, vehicle information, and global positioning system information by the detection systems.
